(19) United States
(12) Patent Application Publication
(10) Pub. No.: US 2023/0158074 A1
(43) Pub. Date: May 25, 2023

(54) B CELL IMMUNOMODULATORY THERAPY FOR ACUTE RESPIRATORY DISTRESS SYNDROME

(71) Applicants: The General Hospital Corporation, Boston, MA (US); Holy Cross Hospital, Inc., Fort Lauderdale, FL (US)

(72) Inventors: Mark C. POZNANSKY, Newton Center, MA (US); Ruxandra SIRBULESCU, Brighton, MA (US); Dusan HANIDZIAR, Brookline, MA (US)

(21) Appl. No.: 17/919,144

(22) PCT Filed: Apr. 16, 2021
(86) PCT No.: PCT/US2021/027727

§ 371 Date: Oct. 14, 2022

Related U.S. Application Data
(60) Provisional application No. 63/010,760, filed on Apr. 16, 2020.

(51) Int. Cl. A61K 35/17; A61P 11/00; A61P 31/16; A61P 31/14; A61K 45/06

(52) U.S. Cl. CPC A61K 35/17; A61P 11/00; A61P 31/16; A61P 31/14; A61K 45/06

(57) ABSTRACT

Disclosed are methods of treating Acute Respiratory Distress Syndrome using B cells, e.g., by administering B cells to a subject in need thereof.

### FIELD OF THE INVENTION

The invention relates to methods of treating Acute Respiratory Distress Syndrome (ARDS).

### BACKGROUND OF THE INVENTION

Acute Respiratory Distress Syndrome (ARDS) results from direct or indirect acute lung injury (ALI) leading to intense inflammation with alveolar edema producing respiratory failure. ARDS accounts for 10% of intensive care units admissions and for 24% of mechanically ventilated subjects. In the United States alone, it affects approximately 200,000 subjects per year with a high mortality rate ranging from 35% to 46%, with higher mortality being associated with greater initial ALI. Further, survivors of ARDS suffer from significant long-term morbidity affecting quality of life with physical, neuropsychiatric, and cognitive impairments. Besides oxygen and mechanical ventilatory support, there are no treatment options currently available and all trials with pharmacologic agents have shown neutral or even deleterious effects.

There is a need for new therapies for treating ARDS that could reduce intensive care unit admissions, mechanical ventilation, and death rate.

### SUMMARY OF THE INVENTION

Provided herein are compositions including B cells (e.g., isolated, purified, or modified B cells or a combination thereof) and uses thereof for the treatment of Acute Respiratory Distress Syndrome.

In one aspect, the invention provides a method of treating Acute Respiratory Distress Syndrome (ARDS) in a subject, the method including administering to the subject a therapeutically effective amount of isolated B cells.

In some embodiments of the preceding aspect, the subject has a viral infection. In some embodiments of any of the preceding aspects, the viral infection is caused by an influenza virus. In some embodiments of any of the preceding aspects, the viral infection is a severe acute respiratory syndrome coronavirus 2 (SARS-CoV-2) infection. In some embodiments of any of the preceding aspects, the subject has been diagnosed with COVID-19.

In some embodiments of any of the preceding aspects, the subject has a bacterial infection.

In some embodiments of any of the preceding aspects, allogeneic B cells are administered. In some embodiments of any of the preceding aspects, autologous B cells are administered. In some embodiments of any of the preceding aspects, xenogeneic B cells are administered.

In some embodiments of any of the preceding aspects, the B cells are mature naïve B cells.

In some embodiments of any of the preceding aspects, the B cells are stimulated ex vivo. In some embodiments of any of the preceding aspects, the B cells are stimulated with a Toll-like receptor (TLR) agonist.

In some embodiments of any of the preceding aspects, the B cells are B_{reg} cells.

In some embodiments of any of the preceding aspects, the B_{reg} cells express immunomodulatory cytokine IL-10.

In some embodiments of any of the preceding aspects, the B_{reg} cells comprise at least 80% CD19+ B cells.

In some embodiments of any of the preceding aspects, the B_{reg} cells comprise less than 10% CD138+ plasma B cells.

In some embodiments of any of the preceding aspects, the B cells are formulated to be administered locally. In some embodiments of any of the preceding aspects, the B cells are formulated to be administered systemically.

In some embodiments of any of the preceding aspects, the B cells are formulated to be administered intravenously.

In some embodiments of any of the preceding aspects, the B cells are administered once daily, once weekly, twice weekly, once every 14 days, or once monthly.

In some embodiments of any of the preceding aspects, the therapeutically effective amount includes at least 0.5×10⁷, at least 1×10⁸, at least 2×10⁸, or at least 1×10⁹ B cells per administration.

In some embodiments of any of the preceding aspects, ARDS results from hyperoxia.

In some embodiments of any of the preceding aspects, the subject has sepsis, pneumonia, or trauma. In some embodiments of any of the preceding aspects, the subject has a tissue injury, inhaled harmful fumes or smoke, inhaled vomited stomach contents from the mouth, or organ failure.

In some embodiments of any of the preceding aspects, treatment results in a decrease in one or more symptoms related to ARDS.

In some embodiments of any of the preceding aspects, the one or more symptoms related to the ARDS is selected from the group consisting of a feeling that one cannot get enough air into the lungs, rapid breathing, low oxygen levels in the blood, and clicking, bubbling, or rattling sounds in the lungs when breathing.

In some embodiments of any of the preceding aspects, isolated B cells are administered in combination with a second therapeutic.

In some embodiments of any of the preceding aspects, the second therapeutic is an antiviral drug, an antimalarial drug, an anti-inflammatory drug, an antibiotic, an acid-reducing medicine, a blood thinner, a muscle relaxant, a pain reliever, a sedative, or a diuretic.

In some embodiments of any of the preceding aspects, the antiviral drug is remdesivir.

In some embodiments of any of the preceding aspects, the second therapeutic is an anti-inflammatory drug.

In some embodiments of any of the preceding aspects, the anti-inflammatory drug is a steroid, parenteral immunoglobulin, or aspirin.

In some embodiments of the preceding aspect, the steroid is dexamethasone.

In some embodiments of any of the preceding aspects, the second therapeutic is convalescent plasma from a subject who has recovered from a coronavirus infection.

In some embodiments of any of the preceding aspects, the subject is a human.

Some additional embodiments of the technology and methodologies described herein are defined according to any of the following numbered paragraphs.

1. A method of treating Acute Respiratory Distress Syndrome (ARDS) in a subject, the method comprising administering to the subject a therapeutically effective amount of isolated B cells.
2. The method of paragraph 1, ARDS results from hyperoxia.
3. The method of paragraph 1, wherein the subject has a viral infection.
4. The method of paragraph 3, wherein the viral infection is caused by an influenza virus.
5. The method of paragraph 3, wherein the viral infection is a SARS-CoV-2 infection.
6. The method of paragraph 3, wherein the subject has been diagnosed with COVID-19.
7. The method of paragraph 1, wherein the subject has a bacterial infection.
8. The method of any one of paragraphs 1-7, wherein allogeneic B cells are administered.
9. The method of any one of paragraphs 1-7, wherein autologous B cells are administered.
10. The method of any one of paragraphs 1-7, wherein xenogeneic B cells are administered.
11. The method of any one of paragraphs 1-10, wherein the B cells are mature naïve B cells.
12. The method of any one of paragraphs 1-11, wherein the B cells are stimulated ex vivo.
13. The method of any one of paragraphs 1-12, wherein the B cells are stimulated with a TLR agonist.
14. The method of any one of paragraphs 1-13, wherein the B cells are B_{reg} cells.
15. The method of paragraph 14, wherein the B_{reg} cells express immunomodulatory cytokine IL-10.
16. The method of paragraph 14 or 15, wherein the B_{reg} cells comprise at least 80% CD19+ B cells.
17. The method of any one of paragraphs 14-16, wherein the B_{reg} cells comprise less than 10% CD138+ plasma B cells.
18. The method of any one of paragraphs 1-17, wherein the B cells are formulated to be administered locally.
19. The method of any one of paragraphs 1-17, wherein the B cells are formulated to be administered systemically.
20. The method of any one of paragraphs 1-17, wherein the B cells are formulated to be administered intravenously.
21. The method of any one of paragraphs 1-20, wherein the B cells are administered once daily, once weekly, twice weekly, once every 14 days, or once monthly.
22. The method of any one of paragraphs 1-21, wherein the therapeutically effective amount comprises at least 0.5×10⁷ B cells per administration.
23. The method of any one of paragraphs 1-22, wherein the therapeutically effective amount comprises at least 1×10⁸ B cells per administration.
24. The method of any one of paragraphs 1-23, wherein the therapeutically effective amount comprises at least 2×10⁸ B cells per administration.
25. The method of any one of paragraphs 1-24, wherein the therapeutically effective amount comprises at least 1×10⁹ B cells per administration.
26. The method of any one of paragraphs 1-25, wherein the subject has sepsis, pneumonia, or trauma.
27. The method of any one of paragraphs 1-26, wherein the subject has a tissue injury, inhaled harmful fumes or smoke, inhaled vomited stomach contents from the mouth, or organ failure.
28. The method of any one of paragraphs 1-27, wherein treatment reduces morbidity or mortality in the clinical course of ARDS, reduces symptoms caused by ARDS, or reduces the need for ventilator dependency.
29. The method of any one of paragraphs 1-28, wherein treatment results in a decrease in one or more symptoms related to ARDS.
30. The method of paragraph 29, wherein the one or more symptoms related to the ARDS is selected from the group consisting of a feeling that one cannot get enough air into the lungs, rapid breathing, low oxygen levels in the blood, and clicking, bubbling, or rattling sounds in the lungs when breathing.
31. The method of any one of paragraphs 1-30, wherein isolated B cells are administered in combination with a second therapeutic.
32. The method of paragraph 31, wherein the second therapeutic is an antiviral drug, an antimalarial drug, an anti-inflammatory drug, an antibiotic, an acid-reducing medicine, a blood thinner, a muscle relaxant, a pain reliever, a sedative, or a diuretic.
33. The method of paragraph 32, wherein the antiviral drug is remdesivir.
34. The method of paragraph 32, wherein the second therapeutic is an anti-inflammatory drug.
35. The method of paragraph 34, wherein the anti-inflammatory drug is a steroid, parenteral immunoglobulin, or aspirin.
36. The method of paragraph 35, wherein the steroid is dexamethasone.
37. The method of paragraph 32, wherein the second therapeutic is convalescent plasma from a subject who has recovered from a coronavirus infection.
38. The method of any one of paragraphs 1-37, wherein the subject is a human.

Other objects, features and advantages of the present invention will become apparent from the following detailed description. It should be understood, however, that the detailed description and specific examples, while indicating preferred embodiments of the invention, are given by way of illustration only, since various changes and modifications within the scope and spirit of the invention will become apparent to one skilled in the art from this detailed description.

### BRIEF DESCRIPTION OF THE DRAWINGS

FIG. 1 is a schematic showing the experimental design testing the effects of B cell immunomodulatory therapy in an in vivo model of acute respiratory distress (ARDS). Mice were exposed continuously for 96 hours to a fraction of inspired oxygen (FiO₂) of greater than 90% (hyperoxia) followed by intravenous (i.v.) administration of 10 million B cells or a saline control solution at 24 hours. Weight assessments were performed daily, at the same time of day, by an experimenter who was blinded to the treatment conditions. At 96 hours, the lungs of mice were biopsied.

FIG. 2 is a graph showing the differential change in the weight of mice treated with B cells or saline control, as normalized to their initial weight, and averaged across the 96 hours of hyperoxia, as described in FIG. 1.

FIG. 3 are photographs of the chest, including the lungs, of mice treated for 96 hours to hyperoxia and treated with B cells or saline control at 24 hours, as described in FIG. 1.

FIG. 4 is a schematic of the lung injury scoring approach, as represented by a set of representative photomicrographs, each depicting a lung injury score (1, 2, 3, and 4, respectively). For example, a score of 0 represents a normal lung, while a score of 4 represents pathological lungs (e.g., widespread cellular infiltration and gross disruption of alveolar architecture). Images of the biopsied lungs of mice exposed to hyperoxia and treated with B cells or saline, as described in FIG. 1, were stained for hematoxylin and scored for lung injury.

FIG. 5 is a graph showing the quantification of the evaluation of lung injury scoring, as described in FIG. 4.

FIG. 6 are photomicrographs of the blood vessels in hematoxylin-stained sections of the lungs of mice exposed to hyperoxia and treated with B cells or saline, as described in FIG. 1.

FIG. 7 is a graph showing the quantification of hematoxylin-positive pixels, indicative of cellular infiltration, in the biopsied lungs of mice exposed to hyperoxia and treated with B cells or saline, as described in FIG. 1, or healthy (no hyperoxia) controls.

FIG. 8 is a graph showing the quantification of B220-stained sections of the lungs of mice exposed to hyperoxia and treated with B cells or saline, as described in FIG. 1, or healthy (no hyperoxia) controls.

### DETAILED DESCRIPTION

The invention will now be described in detail by way of reference only using the following definitions and examples. All patents and publications referred to herein are expressly incorporated by reference. Unless defined otherwise herein, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. Numeric ranges are inclusive of the numbers defining the range.

The headings provided herein are not limitations of the various aspects or embodiments of the invention which can be had by reference to the specification as a whole. Accordingly, the terms defined immediately below are more fully defined by reference to the specification as a whole.

In general, the invention provides methods of treating Acute Respiratory Distress Syndrome (ARDS) in a subject using a therapeutically effective amount of isolated B cells. We describe the application of immunomodulatory B lymphocytes (B cells) or subpopulations of B cells as a therapeutic strategy to mitigate ARDS, such as, for example, those associated with the COVID-19 disease or other infectious causes of ARDS, for example bacterial infections.

### Definitions

As used herein, the terms “Acute Respiratory Distress Syndrome” and “ARDS” refer to a condition that occurs when fluid fills the air sacs in the lungs causing low blood oxygen. ARDS is thus a syndrome characterized by impaired gas exchange resulting in low oxygen tensions in the blood (e.g., hypoxemia) and tissues (e.g., hypoxia). In ARDS, fluid builds inside the air sacs of the lungs, and surfactant breaks down. Surfactant is a foamy substance that keeps the lungs fully expanded so that a person can breathe. These changes prevent the lungs from filling property with air and moving enough oxygen into the bloodstream and consequently throughout the body. The lung tissue may scar and become stiff, further impairing lung function. ARDS may develop over a few days, or it can worsen quickly. The first symptom of ARDS, typically, is shortness of breath. Other signs and symptoms of ARDS include low blood oxygen, rapid breathing, and clicking, bubbling, or rattling sounds in the lungs when breathing.

As used herein, the term “anti-inflammatory” refers to the property of preventing, inhibiting, or reducing inflammation. For example, a composition or method that is anti-inflammatory may be characterized by an alteration (e.g., a reduction) in a symptom associated with an inflammatory disorder. Alternately, a composition or method that is anti-inflammatory may be characterized by a reduction in an inflammatory marker (e.g., a reduction in pro-inflammatory cytokines) or an increase in an anti-inflammatory marker (e.g., an increase in anti-inflammatory cytokines).

As used herein, the term “immunomodulatory” refers to the property of initiating or modifying (e.g., increasing or decreasing) an activity of a cell involved in an immune response. An immunomodulatory composition or method may increase an activity of a cell involved in an immune response, e.g., by increasing pro-inflammatory markers such as cytokines, and/or may decrease an activity of a cell involved in an immune response, e.g., by decreasing pro-inflammatory markers such as cytokines.

As used herein, the terms “B cell” and “B lymphocyte,” as used interchangeably herein, refer to a type of white blood cell of the small lymphocyte subtype. B cells, unlike the other two classes of lymphocytes, T cells and natural killer cells, express B cell receptors (BCRs) on their cell membrane. BCRs allow the B cell to bind to a specific antigen, against which it will initiate an antibody response. B cells function in the humoral immunity component of the adaptive immune system by secreting antibodies. Additionally, B cells present antigens (they are also classified as professional antigen-presenting cells (APCs)) and secrete cytokines. In mammals, B cells mature in the bone marrow. As used herein, the term “mature B cells” refers to a B cell that has completed the process of B cell maturation, for example, in the bone marrow of a mammal. Mature B cells leave the bone marrow and migrate to secondary lymphoid tissues, where they may interact with exogenous antigen and or T helper cells. The stages of B cell maturation have been well-characterized in the scientific literature and are known to those of skill in the art.

As used herein, the term “naïve B cell” refers to a B cell that has not been exposed to an antigen.

As used herein, the terms “B_{reg} cell” and “B regulatory cell” refer to a type of B cell which participates in immunomodulation and in suppression of immune responses. B_{reg} cells of the disclosure are mature, naïve B cells, expressing characteristic cell surface markers. B_{reg} cells may express one or more of B220, CD1d, CD5, CD19, CD20, CD21, CD22, CD23, CD24, CD25, CD27, CD38, CD44, CD48, CD71, CD73, CD138, CD148, CD274, IgM, IgG, IgA, and IgD. In particular, B_{reg} cells may express cell surface markers including but not limited to B220, CD19, CD20, CD24, IgM, IgD, and CD138. Upon introduction into an injured environment, B_{reg} cells can produce immunomodulatory cytokines including but not limited to IL-2, IL-4, IL-6, IL-10, IL-35, TNF-alpha, TGF-beta, interferon-gamma. In particular, B_{reg} cells are characterized by the production of IL-A0.

As used herein, the term “cytokine” refers to refers to a small protein involved in cell signaling. Cytokines can be produced and secreted by immune cells, such as T cells, B cells, macrophages, and mast cells, and include chemokines, interferons, interleukins, lymphokines, and tumor necrosis factors. As used herein, the term “pro-inflammatory cytokine” refers to a cytokine secreted from immune cells that promotes inflammation. Immune cells that produce and secrete pro-inflammatory cytokines include T cells (e.g., Th cells) macrophages, B cells, and mast cells. Pro-inflammatory cytokines include interleukin-1 (IL-1, e.g., IL-1β), IL-5, IL-6, IL-8, IL-10, IL-12, IL-13, IL-18, tumor necrosis factor (TNF, e.g., TNFα), interferon gamma (IFNγ), and granulocyte macrophage colony stimulating factor (GMCSF).

As used herein, the term “Toll-like receptor (TLR) agonist” refers to ligands that bind to and activate Toll-like receptors (TLRs), leading to downstream TLR cell signaling. TLR agonists are known to those of skill in the art and include endogenous and exogenous ligands. Exemplary endogenous ligands which are TLR agonists include heat shock proteins, necrotic cells or a fragment thereof, oxygen radicals, urate crystals, mRNA, beta-defensin, fibrin, fibrinogen, Gp96, Hsp22, Hsp60, Hsp70, HMGB1, lung surfactant protein A, low density lipoprotein (LDL), pancreatic elastase, polysaccharide fragment of heparan sulfate, soluble hyaluronan, alpha A-crystallin, and CpG chromatin-IgG complex. Exemplary exogenous ligands which are TLR agonists include Pam3CSK4, triacylated lipopeptide, glycosylphosphatidylinositol (GPI)-anchored protein, lipoarabinomannan, outer surface lipoprotein, lipopolysaccharide, cytomegalovirus envelope protein, glycoinositolphospholipids, glycolipids, GPI anchor, Herpes simplex virus 1 or a fragment thereof, lipoteichoic acid, mannuronic acid polymer, bacterial outer membrane porin, zymosan, double-stranded RNA, single-stranded RNA, Poly(I).Poly(C), taxol, flagellin, modulin, imidazoquinolines, antiviral compounds, unmethylated CpG oligodeoxynucleotide, and profilin.

As used herein, the term “treatment” (and variations thereof, such as “treat” and “treating”) refers to clinical intervention in an attempt to alter the natural course of the individual being treated and can be performed either for prophylaxis or during the course of clinical pathology. Desirable effects of treatment include, but are not limited to, preventing the occurrence or recurrence of the syndrome, disease, or disorder (such as those described herein), alleviation of symptoms of such syndromes, diminishment of any direct or indirect pathological consequences of the syndromes, as well as altering an immune response. Additionally, treatment refers to clinical intervention relating to any of the syndromes or conditions described herein.

As used herein, by the term “administering” is meant a method of giving a dosage to a subject. The compositions utilized in the methods described herein can be administered, for example, intravenously, intradermally, percutaneously, intraarterially, intraperitoneally, intrapleurally, intratracheally, intranasally, by inhalation, by injection, by infusion, by continuous infusion, or by catheter. The compositions utilized in the methods described herein can also be administered systemically or locally. The method of administration can vary depending on various factors (e.g., the composition being administered, and the severity of the syndrome, condition, disease, or disorder of immune dysregulation being treated).

The term “subject,” as used herein, refers to a human suffering from or at risk of having ARDS. A subject may be diagnosed as having ARDS (e.g., as caused by hyperoxia). Non-limiting examples of ARDS symptoms include a feeling that one cannot get enough air into the lungs, rapid breathing, low oxygen levels in the blood, and clicking, bubbling, or rattling sounds in the lungs when breathing.

A mammal “in need” of treatment can include, but are not limited to, mammals that have ARDS, a viral infection (e.g., a severe acute respiratory syndrome coronavirus 2 (SARS-CoV-2), a COVID-19 infection, a bacterial infection, sepsis, pneumonia, trauma, tissue injury, alcoholism, or drug overdose; or mammals that have had ARDS, a viral infection (e.g., a severe acute respiratory syndrome coronavirus 2 (SARS-CoV-2), a COVID-19 infection, a bacterial infection, sepsis, pneumonia, trauma, tissue injury, alcoholism, or drug overdose; or mammals with symptoms of ARDS, a viral infection (e.g., a severe acute respiratory syndrome coronavirus 2 (SARS-CoV-2), a COVID-19 infection, a bacterial infection, sepsis, pneumonia, trauma, tissue injury, alcoholism, or drug overdose.

As used herein, the term “hyperoxia” refers to an occurrence when cells, tissues, and/or organs are exposed to an excess supply of oxygen (e.g., at pressures greater than normal atmospheric pressure) or a higher-than-normal partial pressure of oxygen.

An “effective amount” of an agent, e.g., a pharmaceutical formulation, refers to an amount effective, at dosages and for periods of time necessary, to achieve the desired therapeutic or prophylactic result or a specifically state purpose. An “effective amount” can be determined empirically and by known methods relating to the stated purpose.

The term “pharmaceutical composition,” as used herein, represents a composition formulated with a pharmaceutically acceptable excipient, and manufactured or sold with the approval of a governmental regulatory agency as part of a therapeutic regimen for the treatment of disease in a subject.

The terms “isolating” and “isolation” refer to both the physical identification and the isolation of a cell or cell population from a cell culture or a biological sample. Isolating can be performed by applying appropriate cell biology technologies that are either based on the inspection of cell cultures and on the characterization (and physical separation when possible and desired) of cells corresponding to the criteria, or on the automated sorting of cells according to a characteristic such as the presence/absence of antigens and/or cell size (such as by FACS). In some embodiments, the terms “isolating” and “isolation” may comprise a further step of physical separation and/or quantification of the cells, especially by carrying out flow cytometry. Physical separation also includes enrichment for a particular characteristic of the cell or cell population. An “isolated” cell or population of cells is a cell or population of cells that has been identified and/or separated as described above.

The terms “cell population” and “population of cells” refer generally to a group of cells. Unless indicated otherwise, the term refers to a cell group consisting essentially of or comprising cells as defined herein. A cell population may consist essentially of cells having a common phenotype or may comprise at least a fraction of cells having a common phenotype. Cells are said to have a common phenotype when they are substantially similar or identical in one or more demonstrable characteristics, including but not limited to morphological appearance, the level of expression of particular cellular components or products (e.g., RNA or proteins), activity of certain biochemical pathways, proliferation capacity and/or kinetics, differentiation potential and/or response to differentiation signals or behavior during in vitro cultivation. Such demonstrable characteristics may therefore define a cell population or a fraction thereof. A cell population may be “substantially homogeneous” if a substantial majority of cells have a common phenotype. A “substantially homogeneous” cell population may comprise at least 60%, e.g., at least 70%, at least 80%, at least 90%, at least 95%, or even at least 99% of cells having a common phenotype, such as the phenotype specifically of a B cell (e.g., a B_{reg} cell). Moreover, a cell population may consist essentially of cells having a common phenotype such as the phenotype of B cell (e.g., a B_{reg} cell) if any other cells present in the population do not alter or have a material effect on the overall properties of the cell population and therefore it can be defined as a cell line. Thus, an isolated cell population (or, for example, isolated B cells) typically comprises at least 60%, or between 60% and 99%, or between 70% and 90%, of B cells (or subpopulations of B cells such as B_{reg} cells).

### Collection and Isolation of B Cells

Any source of B cells, also known as B lymphocytes, may be used for collection purposes. Such B cells may be derived from the bone marrow, spleen, lymph nodes, blood or other allogeneic tissues that are sources of B cells, as known to one of ordinary skill in the art. Preferred sources of B cells are bone marrow and blood. Preferably, autologous or allogeneic or xenogeneic B cells are collected.

Using sterile techniques, in one embodiment, bone marrow is preferably obtained from the posterior superior ilium. The B cells obtained may be immediately used after isolation and relative purification, may be stored for subsequent use, or may be cultured for a period of time before use. The B cell population in the bone marrow contains pre-pro-B cells, pro-B cells, pre-B cells, immature B cells, and some mature B cells.

In the present application, the term B cell encompasses pre-pro-B cells, pro-B cells, pre-B cells, immature B cells, and mature B cells. From blood or other tissues, B cells can be isolated using standard techniques known to one of ordinary skill in the art.

Methods to obtain B cells or, for example, precursor B cells from heterogeneous cell populations are known. Many of these techniques employ primary antibodies that recognize molecules on the surface of the desired B cells or B cell precursors and use these antibodies to positively select these cells and separate them from unwanted cells. This technique is known as positive selection.

Other techniques commonly employed use primary antibodies that recognize molecules on the surface of the cells to be separated from the desired B cells or B cell precursors. In this manner, molecules on these unwanted cells are bound to these antisera and these cells are removed from the heterogeneous cell population. This technique is known as negative selection.

A combination of positive and negative selection techniques may be employed to obtain relatively pure populations of B cells or precursor B cells. Such populations are referred to as isolated B cells. As is used herein, relatively pure means at least 60% pure, 65% pure, 70% pure, 75% pure, 80% pure, 85% pure, 88% pure, or higher degrees of purity such as at least 90% pure, at least 95% pure, at least 97% pure, or at least 98% to 99% pure.

Numerous techniques are available to one of ordinary skill to separate antibodies bound to cells. Antibodies may be linked to various molecules that provide a label or tag that facilitates separation. In one embodiment, primary antibodies may be linked to magnetic beads that permit separation in a magnetic field. In another embodiment, primary antibodies may be linked to fluorescent molecules that permit separation in a fluorescent activated cell sorter. Fluorescent and magnetic labels are commonly used on primary and/or secondary antibodies to achieve separation. Secondary antibodies which bind to primary antibodies may be labeled with fluorescent molecules that permit separation of cells in a fluorescence activated cell sorter. Alternatively, metallic microbeads may be linked to primary or secondary antibodies. In this manner, magnets may be used to isolate these antibodies and the cells bound to them.

To achieve positive or negative selection, the heterogeneous cell population is incubated with primary antibodies for a time sufficient to achieve binding of the antibodies to the antigen on the cell surface. If the primary antibodies are labeled, separation may occur at this step. If secondary antibodies are employed, then the secondary (anti-primary) antibodies are incubated with the cells bound to the primary antibodies for a time sufficient to achieve binding of the secondary antibodies to the primary antibodies. If the secondary antibody has a fluorescent label, then the cells are sent through a fluorescence activated cell sorter to isolate the labeled antisera bound to the desired cell. If the secondary antibody has a magnetic label, then the selected cell with the primary antibody and secondary antibody-labeled microbeads forms a complex that when passed through a magnet remain behind while the other unlabeled cells are removed along with the cell medium. The positively labeled cells are then eluted and are ready for further processing. Negative selection is the collection of the unlabeled cells that have passed through the magnetic field.

Miltenyi Biotec has developed numerous products for the straightforward magnetic separation of B cells and distinct B cell subsets. B cells can be isolated either straight from whole blood or buffy coat without density gradient centrifugation or erythrocyte lysis, or from peripheral blood mononuclear cells (PBMCs) after density gradient centrifugation. Both positive selection and depletion strategies can be pursued for direct isolation and isolation of B cells according to standard methods.

Thus, in a working embodiment, a subject and a potential donor are HLA (A, B, and DR-B1) tested, for example, by the American Red Cross. Potential donors found to be a haploidentical match to the recipient are taken as useful allogeneic donors. Donors are then subjected to apheresis for separating and collecting B cells. B cell product for infusion is then prepared.

Upon receipt of donor allogeneic mononuclear cells—MNC (A), the apheresis product is enriched for B cells using Miltenyi Biotec CliniMACS® CD19 selection. After platelet wash, the product (up to 4×10¹⁰ total cells and up to 5×10⁹ CD19+ cells per vial of CD19 CliniMACS reagent) are processed for CD19+ cells enrichment using CD19 microbeads separated on LS column. The target fraction is washed, and the infusion media is then Plasma-Lyte A supplemented with 25% HSA (1% Final Concentration).

The methods, in general, for example, include:

### Day 1

a. Donor Apheresis Product received and sampled for Sterility, Cell Count, Viability and Flow Cytometry.
b. Product stored refrigerated overnight.

### Day 2

a. Product removed from refrigerator, mixed well, and left to equilibrate to ambient temperature for 30 minutes. Samples removed for sterility, cell count, viability and Flow Cytometry (DuraClone panel with CD20).
b. Platelet wash performed as per a standard CliniMacs procedure.
c. Beads added and incubated for 30 minutes on a rocker as per standard CliniMacs procedure except that incubation performed at 4° C.
d. Post Bead incubation, 1 Antibody wash performed using chilled (4° C.) medium, and product loaded onto the CliniMacs LS column as per standard CliniMacs procedure:
e. Separation run using CliniMacs Enrichment 1.1 program.
f. CD19 Enriched Target fraction sampled for cell count, flow cytometry, stability and sterility.
g. CD19 depleted (Non-Target fraction) sampled for cell count and flow cytometry.

Isolation of B cells from heterogeneous cell populations and stem cell populations may also involve a negative selection process in which the marrow first undergoes red cell lysis by placing the bone marrow in a hypotonic buffer and centrifuging the red blood cells out of the buffer. The red blood cell debris remains in the supernatant which is removed from the test-tube. The bone marrow derived cells are then resuspended in a buffer that has the appropriate conditions for binding antibody. Alternatively, the bone marrow can be subjected to a density gradient centrifugation. The buffy coat layer containing the bone marrow derived cells is removed from the gradient following the centrifugation. The cells are washed and resuspended in the antibody binding buffer and is then incubated with primary antibodies directed toward stem cells, T cells, granulocytes and monocytes/macrophages (called lineage depletion) followed by positive selection using antibodies toward B cells.

Different B-cell subpopulations can be distinguished on the basis of differential expression of various surface markers and collected accordingly.

### Ex Vivo Stimulation of B Cells

Once isolated, B cells may be treated or stimulated by exposing them to one or more TLR agonists or immunomodulatory cytokine as is described herein. Production of IL-10 producing B_{reg} cells using such ex vivo stimulation is taken useful in the methods and therapeutic strategies described herein.

### Administration

The number of cells to be administered will be related to the area or volume of affected area to be treated, and the method of delivery.

One non-limiting range of B cell number for administration is 10⁴ to 10¹⁴ B cells, depending on the volume of tissue or organ to be treated. Other ranges include 10⁵ to 10¹² B cells and 10⁶ to 10¹⁰ B cells. A pharmaceutical composition including B cells (e.g., isolated, purified, B_{reg}, or modified B cells, or a combination thereof) may include 10⁴ to 10¹⁴ B cells, 10⁵ to 10¹² B cells, or 10⁶ to 10¹⁰ B cells in a single dose.

Individual injection volumes can include a non-limiting range of from 1 μL to 1000 μL, 1 μL to 500 μL, 10 μL to 250 μL, or 20 μL to 150 μL. Total injection volumes per subject range from 10 μL to 10 mL depending on the species, the method of delivery and the volume of the tissue or organ to be treated.

### Pharmaceutical Compositions

The B cells described herein may be incorporated into a vehicle for administration into a subject, such as a human subject suffering from a syndrome, disease, or condition described herein. Pharmaceutical compositions containing B cells can be prepared using methods known in the art. For example, such compositions can be prepared using, e.g., physiologically acceptable carriers, excipients or stabilizers (Remington: The Science and Practice of Pharmacology 22nd edition, Allen, L. Ed. (2013); incorporated herein by reference), and in a desired form, e.g., in the form of aqueous solutions.

The B cells described herein can be administered in any physiologically compatible carrier, such as a buffered saline solution or a solution containing one or more electrolytes (e.g., one or more of sodium chloride, magnesium chloride, potassium chloride, sodium gluconate, or sodium acetate trihydrate). For example, the B cells may be administered in a PlasmaLyte infusion buffer. PlasmaLyte is a family of balanced crystaloid solutions with multiple different formulations available worldwide according to regional clinical practices and preferences. It closely mimics human plasma in its content of electrolytes, osmolality, and pH. PlasmaLyte solutions also have additional buffer capacity and contain anions such as acetate, gluconate, and even lactate that are converted to bicarbonate, CO₂, and water. The advantages of PlasmaLyte include volume and electrolyte deficit correction while addressing acidosis. In preferred embodiments, the infusion buffer is PlasmaLyte A. PlasmaLyte A is a sterile, nonpyrogenic isotonic solution for injections (e.g., intravenous) administration. Each 100 mL of PlasmaLyte A contains 526 mg of Sodium Chloride (NaCl); 502 mg of Sodium Gluconate (C₆H₁₁NaO₇); 368 mg of Sodium Acetate Trihydrate, (C₂H₃NaO₂3H₂O); 37 mg of Potassium Chloride (KCl); and 30 mg of Magnesium Chloride (MgCl₂6H₂O). It contains no antimicrobial agents. The pH is adjusted with sodium hydroxide. The pH is about 7.4 (e.g., 6.5 to 8.0).

Other pharmaceutically acceptable carriers and diluents include saline, aqueous buffer solutions, solvents and/or dispersion media. The use of such carriers and diluents is well known in the art. Other examples include liquid media, for example, Dulbeccos modified eagle's medium (DMEM), sterile saline, sterile phosphate buffered saline, Leibovitz's medium (L15, Invitrogen, Carlsbad, Calif.), dextrose in sterile water, and any other physiologically acceptable liquid.

Dispersions may also be prepared in glycerol, liquid polyethylene glycols, and mixtures thereof and in oils. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. In many cases, it will be preferable to include isotonic agents, for example, sugars or sodium chloride. The solution is preferably sterile and fluid to the extent that easy syringability exists. Preferably, the solution is stable under the conditions of manufacture and storage and preserved against the contaminating action of microorganisms such as bacteria and fungi through the use of, for example, parabens, chlorobutanol, phenol, ascorbic acid, thimerosol, and the like. Solutions of the invention can be prepared by using a pharmaceutically acceptable carrier or diluent and, as required, other ingredients enumerated above, followed by filtered sterilization, and then incorporating the B cells as described herein.

For example, a solution containing a pharmaceutical composition described herein may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. The person responsible for administration will, in any event, determine the appropriate dose for the individual subject. Moreover, for human administration, preparations may meet sterility, pyrogenicity, general safety, and purity standards as required by FDA Office of Biologics standards.

The pharmaceutical composition may also include an excipient to promote cell membrane stability. The infusion medium may be supplemented with, for example, a highly soluble osmolytic protein, such as a highly soluble osmolytic protein with a high molecular weight. Serum proteins, such as Human Serum Albumin (HSA), may be included in a pharmaceutical composition described herein as a medium supplement for maintaining cell membrane stability. HSA includes recombinant albumin. Alternately, human serum may be used to stabilize pharmaceutical compositions including cells.

### Method of Treatment

### Acute Respiratory Distress Syndrome

The first signs and symptoms of ARDS includes a feeling that one cannot get enough air into the lungs, rapid breathing, and low oxygen levels in the blood. Other signs and symptoms of ARDS are, for example, clicking, bubbling, or rattling sounds in the lungs when breathing.

A variety of insults resulting in damage either to the vascular endothelium or to the alveolar epithelium could result in ARDS. ARDS, in some instances, may be caused by hyperoxia, a viral infection (e.g., a coronavirus infection), a bacterial infection, sepsis, pneumonia, trauma, tissue injury, alcoholism, drug overdose, breathing in harmful fumes or smoke, and inhaling vomited stomach contents from the mouth. ARDS tends to develop within 48 hours of the event that caused it and it can worsen rapidly.

Current treatments of ARDS focus on supporting the subject while the lungs heal. The goal of supportive care is to get enough oxygen into the blood and delivered to the body to prevent damage and to remove the injury that caused ARDS to develop. A ventilator is a machine used to open airspaces that have shut down and help with the work of breathing. The ventilator is connected to the subject through a mask on the face or a tube inserted into the windpipe. A subject with ARDS is typically in bed on their back. When oxygen and ventilator therapies are at high levels and blood oxygen is still low, a subject with ARDS is sometimes turned over on their stomach to get more oxygen into the blood. However, high levels of oxygen can also injure the lung (e.g., hyperoxia).

Doctors may administer to an ARDS subject medication to relieve symptoms, treat the underlying cause, or prevent complications from being in a hospital. Such medications may include: acid-reducing medicines to prevent stress ulcers, which can cause bleeding in the intestines; antibiotics to treat or prevent infections; blood thinners, e.g., heparin, to stop blood dots from forming or growing larger; muscle relaxants to help prevent coughing or gagging while on a ventilator or to reduce the amount of oxygen the body needs; pain relievers; sedatives to help relieve anxiety, make it easier to breathe on a ventilator, or lower oxygen needs; and diuretics to increase urination to remove excess fluid from the body to help prevent fluid from building up in the lungs.

In other examples, subjects with COVID-19 are at high risk for ARDS and death from respiratory failure. Approximately 15% of COVID-19 subjects develop ARDS, which can have a mortality rate in the range of 40-60%. ARDS develops in the context of COVID-19 primarily due to an over-activation of multiple branches of the immune system (i.e., cytokine storm). SARS-CoV-2 RNA acts as a recognizable pathogen-associated molecular pattern, leading to a chemokine surge which causes neutrophil influx and cytotoxic T-cell activation, ultimately causing the destruction of the alveolar-capillary walls. In other examples, the ARDS subject may be diagnosed as having a coronavirus infection (e.g., COVID-19). Therefore, it is useful to develop therapeutic strategies such as those described herein to mitigate the hyperinflammatory response in COVID-19, without inducing a blanket impairment of the immune system, as immunosuppressant drugs are typically likely to produce.

Advantageously, administration of B cells described herein may reduce morbidity or mortality in the clinical course of ARDS, reduce one or more symptoms caused by ARDS, or reduce the need for ventilator dependency. B cells are a powerful and adaptable immunoregulatory cell type. In contrast to a drug, cell therapy uses dynamic and polyfunctional cells that are responsive to the environment to which they are exposed and exert a multi-modal therapeutic effect.

Beyond their role in the production of antibodies, B-lymphocytes are some of the earliest and strongest modulators of inflammation through their production of IL-10, providing a regulatory break after immune challenge. A variety of B-cells can adopt a regulatory phenotype upon stimulation. Through production of IL-10 and TGFβ, B-cells can convert naive CD4+ T-cells into regulatory T-cells, another subtype of anti-inflammatory lymphocytes that are notably associated with positive outcomes in ARDS. Purified naive B-cells respond to an inflammatory environment by converting to an immunomodulatory phenotype, which can change trafficking and cytokine response of other immune cells, particularly neutrophils and monocytes/macrophages—all of which have been implicated in the development of ARDS.

Unlike other cell types used therapeutically, such as stem cells, B lymphocytes are mature, terminally-differentiated cells, with a defined life span in vivo, thus precluding the need to further manipulate the cells in order to ensure that they do not survive or differentiate off-target over the long term in recipient tissues.

Furthermore, live purified allogeneic B cells or subpopulations of immunoregulatory B cells isolated from peripheral blood may be administered to subjects with ARDS intravenously in order to reduce the overreaction of the immune system (i.e., cytokine storm) to pathogen-induced cell death in the lung.

### EXAMPLES

The following are examples of the methods of the invention. It is understood that various other embodiments may be practiced, given the general description provided above.

### Example 1. Materials and Methods

### Mice

Mice were purchased from Jackson Laboratory. Donor animals for B cell isolation were C57BL/6J mice also purchased from the Jackson Laboratory (Stock No: 000664). All animal procedures were performed following the NIH Guide for Care and Use of Laboratory Animals and the Public Health Service Policy on Humane Care of Laboratory Animals.

### Acute Respiratory Distress Syndrome Model

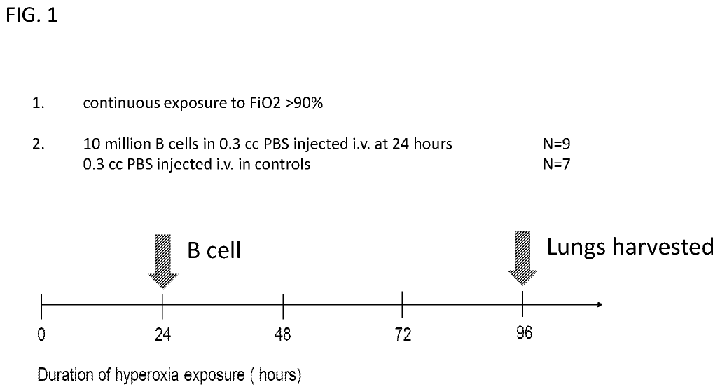

A well-established standardized model of Acute Respiratory Distress Syndrome (ARDS) in mice was used to test the efficacy of B cell immunomodulatory therapy. Mice were exposed continuously for 96 hours to a fraction of inspired oxygen (FiO₂) of greater than 90% (e.g., hyperoxia) (FIG. 1). After 96 hours of FiO₂ exposure, the lungs of mice were biopsied for histology and immunohistochemistry analyses.

### Safety Assessment

Three times per week the health status of all the animals was assessed for potential detrimental effects of the treatment as per IACUC guidelines (apathy, poor body condition, ruffled fur, hunched posture). The weight of mice was measured daily.

### B Cell Isolation and Administration

All cell isolation procedures were performed under sterile conditions, in a clean biosafety cabinet, and the resulting cell suspension was delivered in sterile phosphate-buffered saline (PBS). Cells were isolated and purified from the spleens of C57BL6 wild-type donor animals, sharing half of the genetic background of the recipient (similar to a sibling). Spleens were dissociated into a splenocyte suspension, and commercially available kits (EasySep™ Mouse B Cell Isolation Kit: Catalog #19854, STEMCELL Technologies) were used to isolate all B cells by negative immunomagnetic selection as in our previously published protocols (e.g., see DeKosky et al. (2013) Nat Rev Neural 9, 192-200; Stocchetti et al. (2017) Lancet Neurol 16, 452-464). The resulting cells were >98% CD19+ B cells and were typically over 85-90% CD19+/B220+/IgM+/IgD+, including approximately 5% CD138+ plasma cells, and <1% other cell populations, as confirmed after isolation through flow cytometric analysis (e.g., see DeKosky et al. (2013) Nat Rev Neural 9, 192-200; Sirbulescu et al. (2017) Wound Repair Regen. 25(5):774-791). This represented the naive B cell fraction (treatment), which was infused into animals the same day, after isolation. After 24 hours of exposure to a FiO₂ of greater than 90% (i.e., hyperoxia), mice received a single intravenous (i.v.) injection of 10 million B cells in 0.3 cc PBC or 0.3 cc PBS (saline control).

### Histology and Immunohistochemistry

Biopsied lungs were fixed in 10% formalin overnight, then preserved in 70% ethanol. The tissue was embedded in paraffin and cut in 5-μm sections.

For histologic analysis, sections were stained with hematoxylin or immunohistochemical staining was performed. Tissues were permeabilized and slides were washed and blocked for 30 min with 5% goat serum. Sections were incubated with primary antibody B220 (BioLegend) for 1 hour at room temperature. Slides were incubated with secondary antibody for 30 min. After washing with PBS, the sections were incubated in 3,3′-diaminobenzidine (Vector Laboratories) for 10 min. Cells were counterstained with Gill's #2 hematoxylin for 10-15 s. Stained tissue sections were images using a transmitted light microscope equipped with a camera. Assessments included detailed standardized pathological scoring of lung injury (criteria defined by the American Thoracic Society) or hematoxylin densitometry quantification with ImageJ software. All counts were performed by an experimenter blinded to the treatment conditions.

### Example 2. B Cell Administration is Protective in a Model of Hyperoxia-Induced Acute Respiratory Distress Syndrome

This Example illustrates the safety and efficacy of i.v. B cell administration in a murine model of ARDS.

### Materials and Methods

Materials and Methods are described in Example 1.

### Results

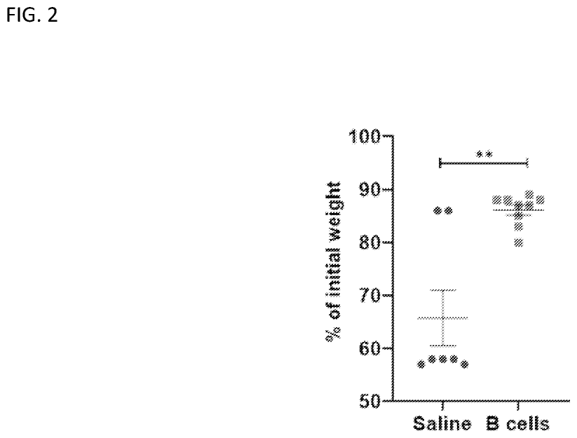

The weight of mice was measured at 96-hour exposure to hyperoxia. FIG. 2 shows the weight of animals in one measurement after four days of exposure to hyperoxia and treatment with either B cells or a control saline solution. Mice treated with B cells displayed a significantly larger weight, as normalized to the percent of their initial weight, demonstrating that B cell administration is protective of ARDS-associated weight loss in a murine model of hyperoxia-induced ARDS.

### Example 3. B Cell Administration Leads to Macroscopic and Histological Protection of the Lung Tissue

This Example describes the ex vivo analyses of B cell immunomodulatory therapy for the prevention and treatment of ARDS-associated pathophysiology, for example, in a murine model of ARDS.

### Materials and Methods

Materials and Methods are described in Example 1.

### Results

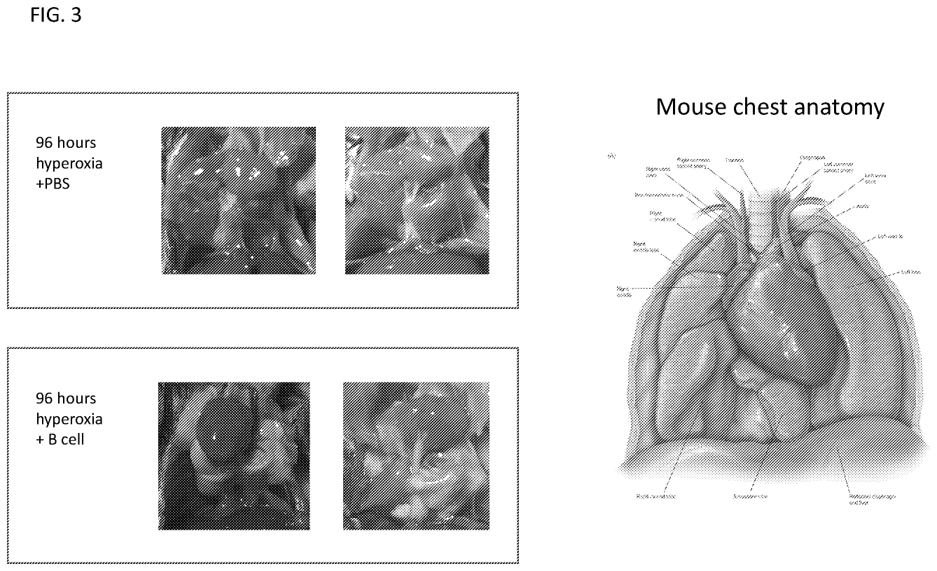

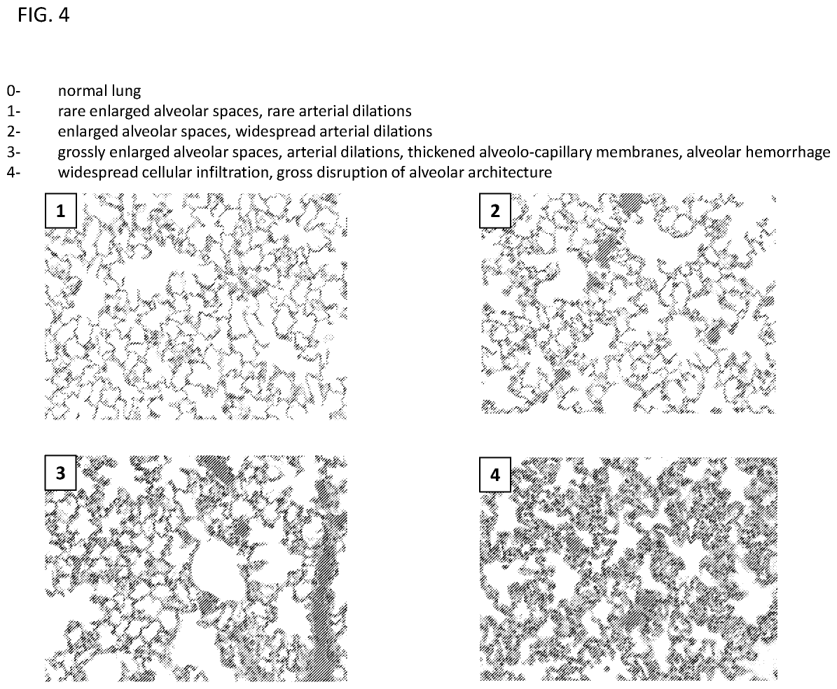

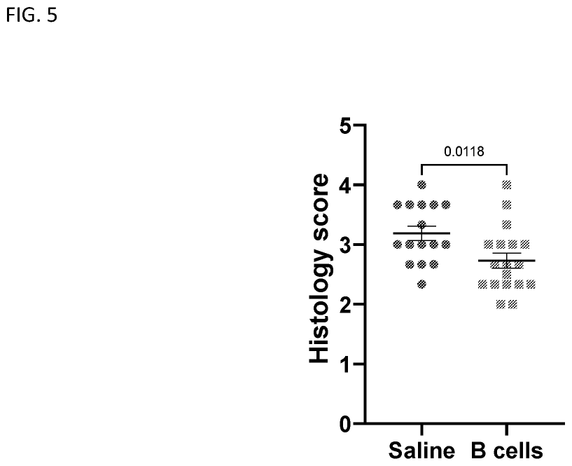

Using the same mice as described in Example 2 for ex vivo analyses, when lungs were biopsied, macroscopic differences in the lungs were observed. While control mice exhibited diffuse lung congestion following 96-hour hyperoxia, B cell-treated mice displayed only regional lung congestion (FIG. 3). To score this ARDS-induced lung pathology with a standardized approach, we used the scoring criteria defined by the American Thoracic Society. Specifically, the images of biopsied lungs were scored on a scale of 0-4, with a score of 0 defined for normal lungs and a score of 4 defined for highly pathological lungs (FIG. 4). FIG. 5 is the quantification of this scoring criteria, as applied to the images of lungs of mice exposed to hyperoxia for 96 hours and treated with B cells or a control solution at 24 hours. B cell-treated mice displayed significantly lower histology scores (Mann Whitney test; N=16 saline/18 B-cell). Taken together, these results support the finding that systemic treatment with B cells reduces ARDS-associated lung pathophysiology and enables histological projection of the lung tissue.

### Example 4: Cellular Density of Infiltrate is Decreased in the Lungs of Mice Treated with B Cells

This Example describes the ex vivo analyses of B cell immunomodulatory therapy for the treatment of ARDS-associated inflammatory cell infiltration, for example, in a murine model of ARDS.

### Materials and Methods

Materials and Methods are described in Example 1.

### Results

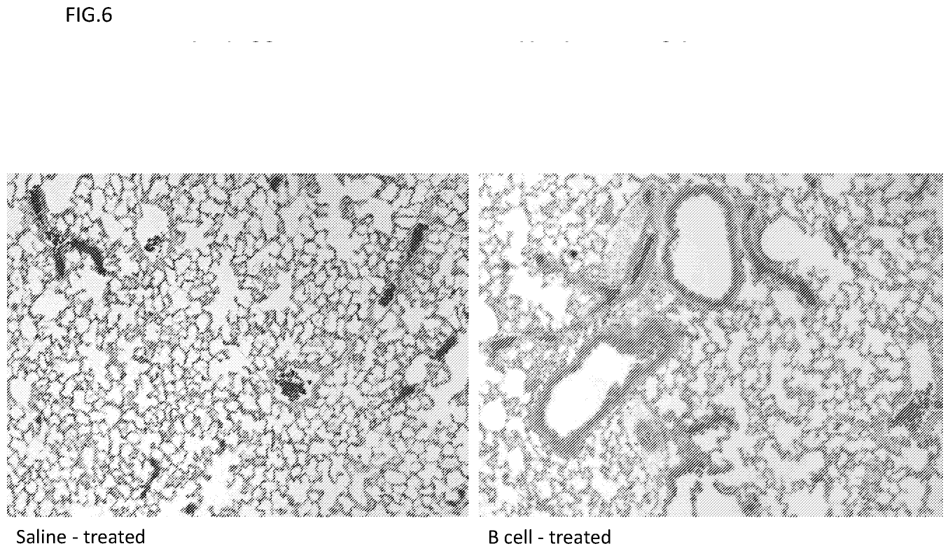

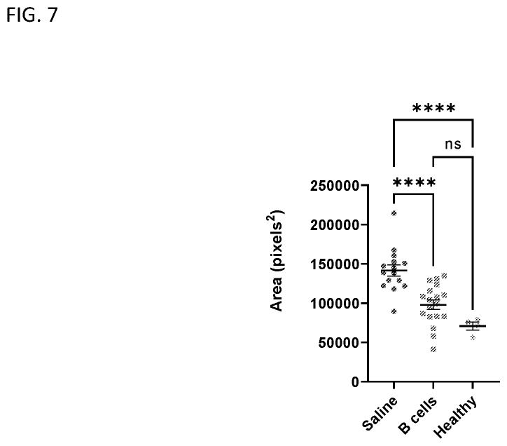

Using the same mice as described in Example 2 for ex vivo analyses, when lungs were biopsied, differences in ARDS-associated lung pathophysiology were observed, as determined with nuclear staining of hematoxylin to assess the levels of cellular infiltration. Specifically, these differences in cellular infiltration were apparent within blood vessels of the lungs of B cell-treated mice, as compared to saline-treated controls (FIG. 6). To mediate an unbiased quantification approach of these data, ImageJ software was used to perform densitometry analyses, such that the number of hematoxylin-positive pixels were counted. FIG. 7 is a quantification of hematoxylin-positive pixels in mice exposed to hyperoxia and treated with B cells or saline, or no-hyperoxia healthy controls. The number of hematoxylin-positive pixels were restored to healthy levels in mice treated with B cells. In contrast, hyperoxia-exposed mice treated with a control saline solution had significantly greater hematoxylin-positive pixels, as compared to B cell-treated hyperoxia-exposed mice and healthy controls. These results further support the finding that systemic treatment with B cells reduces ARDS-associated lung pathophysiology, including inflammatory cell infiltration

### Example 5: Intravenous B Cell Administration Rescues the Hyperoxia-Induced Reduction of B220-Positive B Cells in the Lung

This Example describes the efficacy of i.v. B cell administration in a murine model of ARDS for the attenuation of ARDS-induced loss of B220-positive B cells in the lungs.

### Materials and Methods

Materials and Methods are described in Example 1.

### Results

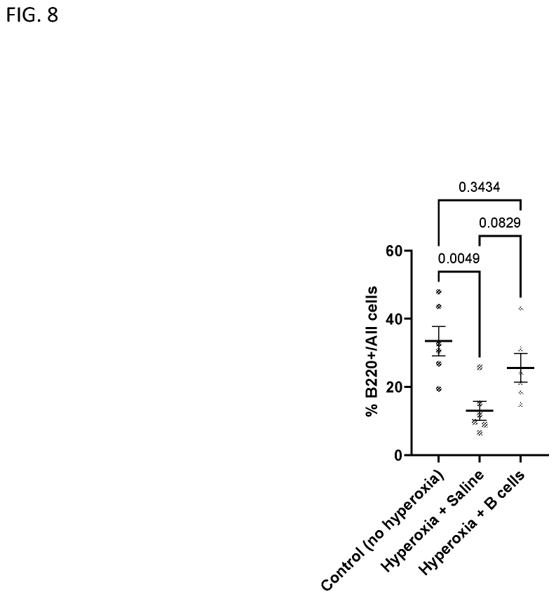

Using the same mice as described in Example 2 for ex vivo analyses, following exposure to hyperoxia and treatment with B cells or a control saline solution, the lungs of mice were biopsied and B220-positive B cells were quantified. FIG. 8 is a graph showing the quantification of said B220-positive B cells in the lungs of B cell-treated, saline-treated controls, and healthy (no hyperoxia) control mice. The number of B220-positive cells were restored to healthy levels in mice treated with B cells. In contrast, hyperoxia-exposed mice treated with a control saline solution had significantly less B220-positive cells, as compared to B cell-treated hyperoxia-exposed mice and controls (no hyperoxia). Taken together, these results demonstrate that hyperoxia leads to a significant reduction in B220-positive B cells in the lung, which can be rescued by intravenous B cell administration.

### Example 6. Administration of B Cells to Treat Acute Respiratory Distress Syndrome

A composition including a therapeutically effective amount of B cells, such as any of the compositions described herein, may be administered to a subject having ARDS. Treatment of ARDS may be evaluated using the methods described herein by administering therapeutic B cells (isolated, purified, B_{reg}, or modified B cells, or a combination thereof) to an appropriate animal model for ARDS (e.g., see Example 1) and monitoring the therapeutic efficacy according to methods known to those of skill in the art. Methods for monitoring the response include assessment of breathing (e.g., shortness of breath) and hemodynamic monitoring.

Responsiveness to treatment may be monitored by a decrease in the rate of progression of the syndrome (e.g., a decrease in the rate of progression as measured by the severity of symptoms associated with ARDS). Alternately, responsiveness to treatment may be monitored by determining the level of a molecular marker of syndrome progression associated with ARDS, such as reduction of B220-positive B cells in the lung.

### Example 7. B Cell Immunomodulatory Therapy in an Animal Model of Viral-Induced ARDS

Using the aforementioned model of ARDS in mice the safety and efficacy of B-cell immunomodulatory therapy is evaluated. Mice (males and females; N=20/condition) are inoculated intranasally with 10,000 PFU mouse-adapted H1N1 influenza virus (Charles River), which has been shown previously to induce ARDS within 4-6 days of administration. At day 4 after inoculation with live virus or control, mice are to receive a single dose of 10 million B-cells either intravenously or by oropharyngeal administration. Control animals are to receive similar volumes of saline solution. Two days later (day 6 post-viral inoculation, when the most acute ARDS pathology is observed), the animals will be euthanized, and peripheral blood, spleen and lymph nodes, bronchoalveolar lavage (BAL), and lung tissues will be collected. Assessments will include levels of pro-inflammatory cytokines (e.g., IL-6, TNFa) in blood plasma, immune cell phenotyping of blood, lymph nodes, spleen, and BAL using 21-color flow cytometry, and pathological scoring of lung injury as described above.

### Example 8. Administration of B Cells to Treat COVID-19

A composition including a therapeutically effective amount of B cells, such as any of the compositions described herein, may be administered to a subject having COVID-19-associated ARDS. Treatment of ARDS may be evaluated using the methods described herein by administering therapeutic B cells (isolated, purified, B_{reg}, or modified B cells, or a combination thereof) and monitoring the therapeutic efficacy according to methods known to those of skill in the art.

Methods for monitoring the response include assessment of breathing (e.g., shortness of breath) and hemodynamic monitoring. Responsiveness to treatment may be monitored by a decrease in the rate of progression of the syndrome (e.g., a decrease in the rate of progression as measured by the severity of symptoms associated with ARDS).

Positive results preclinical studies will establish the necessary rigorous foundation for the rapid translation towards first in human testing. Given that purified mature B cells can be easily accessed from peripheral blood, a rapid, minimally manipulated, allogeneic B-cell therapy is translatable to a clinical setting, posing significantly lower regulatory barriers than any other available cell-based therapy that requires cell manipulation. Pre-banked HLA-typed B-cell product from healthy donors may be, accordingly, administered to subjects with COVID-19 ARDS to prevent deleterious symptom progression and reduce mortality.

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

What is claimed is:

1. A method of treating Acute Respiratory Distress Syndrome (ARDS) in a subject, the method comprising administering to the subject a therapeutically effective amount of isolated B cells.

2. The method of claim 1, wherein ARDS results from hyperoxia.

3. The method of claim 1, wherein the subject has a viral infection.

4. The method of claim 3, wherein the viral infection is caused by an influenza virus.

5. The method of claim 3, wherein the viral infection is a severe acute respiratory syndrome coronavirus 2 (SARS-CoV-2) infection.

6. The method of claim 3, wherein the subject has been diagnosed with COVID-19.

7. The method of claim 1, wherein the subject has a bacterial infection.

8. The method of claim 1, wherein allogeneic B cells are administered.

9. The method of claim 1, wherein autologous B cells are administered.

10. The method of claim 1, wherein xenogeneic B cells are administered.

11. The method of claim 1, wherein the B cells are mature naïve B cells.

12. The method of claim 1, wherein the B cells are stimulated ex vivo.

13. The method of claim 1, wherein the B cells are stimulated with a Toll-like receptor (TLR) agonist.

14. The method of claim 1, wherein the B cells are B_{reg} cells.

15. The method of claim 14, wherein the B_{reg} cells express immunomodulatory cytokine IL-10.

16. The method of claim 14, wherein the B_{reg} cells comprise at least 80% CD19+ B cells.

17. The method of claim 14, wherein the B_{reg} cells comprise less than 10% CD138+ plasma B cells.

18. The method of claim 1, wherein the B cells are formulated to be administered locally.

19. The method of claim 1, wherein the B cells are formulated to be administered systemically.

20. The method of claim 1, wherein the B cells are formulated to be administered intravenously.

21. The method of claim 1, wherein the B cells are administered once daily, once weekly, twice weekly, once every 14 days, or once monthly.

22. The method of claim 1, wherein the therapeutically effective amount comprises at least 0.5×10⁷ B cells per administration.

23. The method of claim 1, wherein the therapeutically effective amount comprises at least 1×10⁸ B cells per administration.

24. The method of claim 1, wherein the therapeutically effective amount comprises at least 2×10⁸ B cells per administration.

25. The method of claim 1, wherein the therapeutically effective amount comprises at least 1×10⁹ B cells per administration.

26. The method of claim 1, wherein the subject has sepsis, pneumonia, or trauma.

27. The method of claim 1, wherein the subject has a tissue injury, inhaled harmful fumes or smoke, inhaled vomited stomach contents from the mouth, or organ failure.

28. The method of any one of claims 1-27, wherein treatment reduces morbidity or mortality in the clinical course of ARDS, reduces symptoms caused by ARDS, or reduces the need for ventilator dependency.

29. The method of claim 28, wherein treatment results in a decrease in one or more symptoms related to ARDS.

30. The method of claim 29, wherein the one or more symptoms related to the ARDS is selected from the group consisting of a feeling that one cannot get enough air into the lungs, rapid breathing, low oxygen levels in the blood, and clicking, bubbling, or rattling sounds in the lungs when breathing.

31. The method of claim 30, wherein isolated B cells are administered in combination with a second therapeutic.

32. The method of claim 31, wherein the second therapeutic is an antiviral drug, an antimalarial drug, an anti-inflammatory drug, an antibiotic, an acid-reducing medicine, a blood thinner, a muscle relaxant, a pain reliever, a sedative, or a diuretic.

33. The method of claim 32, wherein the antiviral drug is remdesivir.

34. The method of claim 32, wherein the second therapeutic is an anti-inflammatory drug.

35. The method of claim 34, wherein the anti-inflammatory drug is a steroid, parenteral immunoglobulin, or aspirin.

36. The method of claim 35, wherein the steroid is dexamethasone.

37. The method of claim 31, wherein the second therapeutic is convalescent plasma from a subject who has recovered from a coronavirus infection.

38. The method of claim 1, wherein the subject is a human.
